(19) United States
(12) Patent Application Publication
(10) Pub. No.: US 2016/0120607 A1
(43) Pub. Date: May 5, 2016

(54) ULTRASONIC IMAGING DEVICE FOR EXAMINING SUPERFICIAL SKIN STRUCTURES DURING SURGICAL AND DERMATOLOGICAL PROCEDURES

(71) Applicants: Michael Sorotzkin, Brooklyn, NY (US); Natalie Lioubashevsky, Bet Hakerem (IL)

(72) Inventors: Michael Sorotzkin, Brooklyn, NY (US); Natalie Lioubashevsky, Bet Hakerem (IL)

(21) Appl. No.: 14/531,745

(22) Filed: Nov. 3, 2014

(51) Int. Cl. A61B 19/00; A61B 8/00; A61B 8/08

(52) U.S. Cl. CPC A61B 19/54; A61B 8/0891; A61B 8/085; A61B 8/463; A61B 8/0825; A61B 8/488; A61B 8/4427; A61B 2019/5408; A61B 2019/545

(57) ABSTRACT

An ultrasonic imaging system for indicating blood vessels of a human body includes an ultrasonic imaging module, a display module, a marking module, and processing module operatively coupled to the ultrasonic imaging module, the display module, and the marking module. The processing module is configured to obtain, using the ultrasonic imaging module, ultrasonic images corresponding to a portion of the human body; present, using the display module, the ultrasonic images, where the ultrasonic images depict one or more blood vessels of the human body; determine that the ultrasonic imaging system is aligned according to a particular blood vessel depicted in the ultrasonic images; and upon determining that the ultrasonic imaging system is aligned according to the particular blood vessel depicted in the ultrasonic images, apply, using the marking module, a marking substance to a surface of the human body. The marking substance is applied to a point immediately above the particular blood vessel.

### TECHNICAL FIELD

This disclosure relates to ultrasonic imaging, and more particularly to ultrasonic imaging devices for use during surgical and dermatological procedures.

### BACKGROUND

Breast cancer refers to cancer that develops from breast tissue. Worldwide, breast cancer is one of the most common invasive cancers in women. The American Cancer Society estimates in 2014, there will be about 232,570 new cases of invasive breast cancer, about 62,570 new cases of carcinoma in situ (CIS) of the breast, and about 40,000 deaths from breast cancer in the United States alone.

Once breast cancer is diagnosed, in many cases a surgical procedure is performed. For example, in some cases, a mastectomy can be carried out in order to remove some or all breast tissue from a breast as a way of treating breast cancer. Further, in some cases, (e.g., for patients at particularly high risk of breast cancer, such as BRCA carriers), a mastectomy can be performed prophylactically as a preventive measure against the onset of breast cancer.

In an example mastectomy procedure, two long horizontal incisions are introduced across the front of a patient's breast. Once the breast tissue is removed, the breast skin is closed as a straight line, leaving a scar across the chest that is the width of the breast. When the volume of the breast is replaced with an implant, the shape is often more bowl shaped than breast shaped due to loss of this skin.

Many patients are sensitive to the cosmetic outcome of a surgery, and different surgical techniques can be used to improve the cosmetic outcome. In many cases, newer mastectomy techniques can preserve breast skin and allow for a more natural breast appearance following the procedure. Surgery to restore shape to breast (e.g., breast reconstruction) may be performed at the same time as a mastectomy or during an additional operation at a later date.

A skin-sparing mastectomy, also known as breast-conserving surgery, is a way to treat cancer or act as a preventive measurement for high risk patients, and save the breast skin. A skin-sparing mastectomy causes less scarring than a traditional mastectomy. In an example skin-sparing procedure, cancerous breast tissue is removed through a small incision of the breast, for example around the areola area of the nipple. The surgeon leaves most of the breast skin, creating a natural skin envelope, or pocket, that is filled with a breast implant or with the patient's own tissue from another part of her body. In many cases, the skin-sparing technique can significantly improve the cosmetic outcome and can provide a better option for subsequent breast reconstruction.

An important aspect of skin-sparing mastectomy is determining whether or not to preserve or sacrifice the nipple or nipple and areola. If the cancer does not involve the nipple or areola, then it may be possible to perform a mastectomy and subsequent breast preservation procedure in a manner that spares the nipple and areola. In cases of preventive mastectomy, the preservation of the nipple and the areola may be a particular importance. However, a common complication of such surgery is skin necrosis (e.g., occurring in up to 20% of cases). Skin necrosis is caused by involuntary transection (e.g., cutting) of superficial skin supplying vessels during the surgical procedure. If a surgeon cuts the superficial skin in a manner that severs vessels that supply the skin, portions of the skin, possibly including the nipple and/or areola may be lost. Complicating matters further, the anatomical location of these blood vessels is patient-specific and can vary greater from one patient to another.

### SUMMARY

In general, in an aspect, a method for indicating blood vessels of a human body includes obtaining, using a handheld ultrasonic imaging device, ultrasonic images corresponding to a portion of the human body. The method also includes identifying, using a display of the ultrasonic imaging device, one or more blood vessels depicted in the ultrasonic images. The method also includes aligning the ultrasonic imaging device according to a particular blood vessel identified in the ultrasonic images. The method also includes applying, using the ultrasonic imaging device, a marking substance to a surface of the human body, where the marking substance is applied to a point immediately above the particular blood vessel.

Implementations of these aspect may include or more of the following features.

In some implementations, aligning the ultrasonic imaging device can include moving the ultrasonic imaging device with respect to the human body until an indicator of the display aligns with the particular vessel identified in the ultrasonic images.

In some implementations, aligning the ultrasonic imaging device can include moving an indicator of the display with respect to the ultrasonic images until the indicator aligns with the particular vessel identified in the ultrasonic images.

In some implementations, the method can further include moving the ultrasonic imaging device along the human body in order to obtain additional ultrasonic images corresponding to different portions of the human body, maintaining the alignment between the ultrasonic imaging device and the particular blood vessel identified in the additional ultrasonic images during movement of the ultrasonic imaging device, and applying, using the ultrasonic imaging device, the marking substance onto the surface of the human body, where the marking substance is applied along a path immediately above the particular blood vessel.

In some implementations, the ultrasonic images can be displayed in real-time on the display of the ultrasonic imaging device.

In some implementations, the method can further include repeating the steps of obtaining, identifying, aligning, and applying until a plurality of blood vessels of the human body are marked. The method can further include incising the human body in a manner that avoids the applied marking substance. Incising the human body can be performed as a part of a mastectomy procedure.

In some implementations, the particular blood vessel can be a skin-supplying blood vessel.

In some implementations, the particular blood vessel can be within approximately 1 cm of a surface of the human body.

In some implementations, the ultrasonic images can be B-mode, color Doppler ultrasonic images.

In general, in another aspect, an ultrasonic imaging system for indicating blood vessels of a human body includes an ultrasonic imaging module, a display module, a marking module, and processing module operatively coupled to the ultrasonic imaging module, the display module, and the marking module. The processing module is configured to obtain, using the ultrasonic imaging module, ultrasonic images corresponding to a portion of the human body, present, using the display module, the ultrasonic images, wherein the ultrasonic images depict one or more blood vessels of the human body, determine that the ultrasonic imaging system is aligned according to a particular blood vessel depicted in the ultrasonic images, and upon determining that the ultrasonic imaging system is aligned according to the particular blood vessel depicted in the ultrasonic images, apply, using the marking module, a marking substance to a surface of the human body. The marking substance is applied to a point immediately above the particular blood vessel.

Implementations of these aspect may include or more of the following features.

In some implementations, determining that the ultrasonic imaging system is aligned according to the particular blood vessel depicted in the ultrasonic images can include receiving user input indicating that the particular blood vessel is aligned with an alignment indicator of the display module. The alignment indicator can include a cursor. The cursor can be configured to move with respect to the ultrasonic images.

In some implementations, determining that the ultrasonic imaging system is aligned according to the particular blood vessel depicted in the ultrasonic images can include identifying, using the processing module, one or more blood vessels depicted in the ultrasonic images, and determining that the ultrasonic imaging system is aligned according to the particular blood vessel based on the identification. Identifying one or more blood vessels depicted in the ultrasonic images can include identifying regions of the ultrasonic images indicative of flowing blood. Identifying one or more blood vessels depicted in the ultrasonic images can include receiving user input indicating a location of a blood vessel.

In some implementations, the marking module can include an applicator configured to apply the marking substance at a pre-defined point relative to the ultrasonic imaging system.

In some implementations, the marking module can include a plurality of applicators each configured to apply the marking substance at a respective pre-defined point relative to the ultrasonic imaging system. The pre-defined points can be arranged as a straight line. The plurality of pre-defined points can be arranged as a grid. Applying a marking substance to a surface of the human body can include selecting a particular applicator to apply the marking substance based on an alignment between the particular applicator and the particular blood vessel. The processor can be configured to present the ultrasonic images in substantially real-time.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a simplified block diagram of an example ultrasonic imaging system.

FIG. 2A shows a front view of an example ultrasonic imaging system.

FIG. 2B shows a bottom view of the example ultrasonic imaging system shown in FIG. 2A.

FIG. 2C shows a side view of the example ultrasonic imaging system shown in FIG. 2A.

FIGS. 3A-D show an example usage of an ultrasonic imaging system.

FIGS. 4A-C show example ultrasonic imaging systems implemented as systems of multiple interconnected devices.

FIG. 5A shows a front view of an example screen.

FIG. 5B shows a bottom view of an example ultrasonic imaging system.

FIG. 6 shows another example ultrasonic imaging system.

FIG. 7 shows an example process of detecting blood vessels.

FIG. 8 shows an example computer system.

### DETAILED DESCRIPTION

Implementations of a system for indicating blood vessels (e g, skin supplying blood vessels) of a human breast are described below. One or more implementations of this system can be used to identify and mark blood vessels that are near the surface of the skin in order to guide a surgeon during a medical procedure. An example, prior to a surgical procedure on a patient (e.g., a mastectomy), a user (e.g., a medical imagine technician, radiologist, or surgeon) can use the system to identify skin supplying blood vessels (e.g., blood vessels that are near the surface of the patient's skin). The user can also use the system to mark the surface of the patient's skin above the location of the identified blood vessels. During the surgical procedure, a surgeon can use the markings to determine the location of the blood vessels, and guide his incisions in a manner that avoids transecting these blood vessels. Thus, use of this system reduces the likelihood that a skin supplying blood vessel is severed during the surgical procedure, and improves the likelihood that the skin, nipple, and/or areola will be preserved. In some implementations, the system is handheld, such that the user can easily acquire images of the patient, identify blood vessels, and mark the identified vessels using the system. In some implementations, the imaging, identification, and marking functionality of the system can be automated or semi-automated, such that the system can mark blood vessels without require extensive user input. Further, some implementations of this system can also be used for prior to surgical procedures other than mastectomies and/or on regions other than a human breast in order to identify skin supplying blood vessels in a variety of contexts (e.g., in the context of plastic surgery and dermatology).

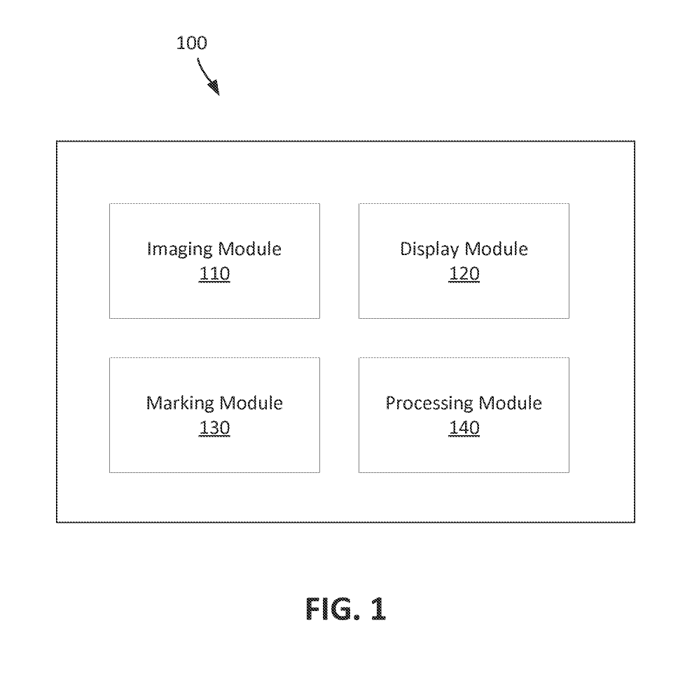

A simplified block diagram of an example ultrasonic imaging system is shown in FIG. 1. The system 100 includes an imaging module 110, a display module 120, a marking module 130, and a processing module 140 operatively coupled to the imaging module 110, the display module 120, and the marking module 130.

The imaging module 110 is an ultrasonic imaging module, and is configured to obtain ultrasonic images of a particular region of the human body. For example, in some implementations, the imaging module 110 includes an ultrasonic transducer that sends pulses of ultrasonic energy into the human body. The ultrasonic energy reflect off parts of the human body (e.g., tissue, organs, and other structures), and a subset of the energy returns of the transducer. The transducer detects the returning energy and generates images based on the detected energy. The imaging module 110 can, for example, operate using in B-mode, which generates two-dimensional cross-sections of the tissue being image. In some implementations, the imaging module 110 can generate images that depict other features and phenomena, such as blood flow, motion of tissue over time, the location of blood, the presence of specific molecules, the stiffness of tissue, or the anatomy of a three-dimensional region.

The display module 120 is a display device that is configured to present graphical information to a user. For example, in some implementations, the display module 120 can present the ultrasonic images obtain by the imaging module 110. In some implementations, the display module 120 can also present a user interface that allows the user to monitor the operation of the system 100. For example, the display module 120 can be used to present the user with one or more visual elements that describe the operational parameters of the system 100 (e.g., the operational parameters being used the imaging module 110 to generate the ultrasonic images), information to guide the user during operation of the system 100 (e.g., an indication of how to align the system 100 with a particular blood vessel, or help or tutorial information that describe how to use the system 100), and notification information (e.g., warnings, alerts, or other information that indicate errors or faults during operation of the system 100). In some implementations, the display module 120 also accepts user input (e.g., using a touchscreen, buttons, switch, or other input mechanisms) that allow the user to interact with the system 100. For example, the display module 120 can allow the user to input or modify operational parameters of the system 100, or otherwise issue commands of the system 100.

The marking module 130 is configured to mark the surface of the skin. For example, the marking module 130 can include an ink-based applicator, such as a pen, stamp, or brush that applies ink to the surface of the skin. In some implementations, the marking module 130 can selectively apply ink. For example, the marking module 130 can include a retractable ink applicator that extends from the system 100 to mark the skin, and retracts after marking is complete. In another example, the marking module 130 can include an applicator that releases ink selectively to mark the skin (e.g., using a pump), and stops releases ink after marking is complete. In some implementations, the marking module 130 can mark a single point of the skin (e.g., using a single applicator). In some implementations, the marking module 130 can mark multiple points or lines (e.g., using multiple applicators in a row, column, or other arrangement). In some implementations, the marking module 130 is positioned such that the images displayed by the display module 120 can be used to align the marking module 130 to particular features depicted in the display module 120. For example, in some implementations, the display module 120 can include a cursor, marker, or other alignment indicator than indicates a particular point or line on the ultrasonic images. In this case, the marking module 130 can be aligned to the cursor, such that the marking module 130 applies ink to the skin at the point indicated by the cursor. Thus, a user can position the system 100 using the display module 120, align the system 100 to a particular feature of interest, and use the marking module 130 to mark the skin above that feature of interest.

The processing module 140 is operably coupled to the imaging module 110, the display module 120, and the marking module 130, and can be used to process information received from each of the modules and/or transmit information to each of the modules so that the modules can perform the functions described above. For example, referring to the imaging module 110, the processing module 140 can control the operation of the imaging module 110 (e.g., by issuing commands that dictate the operation of the transducer of the imaging module 110). The processing module 140 can also interpret information obtained by the imaging module 110 (e.g., by receiving information describing the detected ultrasonic energy, generating ultrasonic images based on this detected energy, and analyze the ultrasonic images in order to identify objects of interest). As another example, referring to the display module 120, the processing module 140 can transmit information to be displayed on the display module 120 (e.g., ultrasonic images, a user interface, and other information), and receive information obtained by the display module 120 (e.g., indications of user input and selections). As another example, referring to the marking module 130, the processing module 140 can control when and where the marking module applies markings. For instance, the processing module 140 can determine when the ultrasonic imaging device is aligned according to a particular blood vessel identified in the ultrasonic images (e.g., by automatically detecting the alignment or receiving user input indicating that the ultrasonic image device is aligned), and in response, control the marking module 130 to apply a marking on the skin above an identified blood vessel.

In an example usage of the system 100, a user (e.g., a medical imaging technician, a radiologist, or a surgeon) uses the imaging module 110 to obtain ultrasonic images of a patient by aligning the imaging module 110 with a particular region of interest. The imaging module 110 transmits ultrasonic energy into the region of interest, and detects returning ultrasonic energy that echoes from features within the region of interest. Based on the detected ultrasonic energy, a series of ultrasonic images are generated and displayed on the display module 120. Using the display module 120, the user observes various features within the region of interest (e.g., blood vessels and other tissue), and identifies a particular blood vessel within the region of interest. In some cases, the blood vessel can appear as a hypoechoic tubular structure (e.g., a circle, oval or ellipse having a particular shade of color). The user then aligns the system 100 to a particular blood vessel, and uses the marking module 130 to mark the surface of the skin above that particular blood vessel. After the skin has been marked, the user can continue using the system 100 to mark other locations of the skin, for example by lifting the system 100 away from the skin and moving it to another location, or sliding the system 100 along the system and tracking the location of the blood vessel using the display module 120. After the user is satisfied with the markings, the device is removed from the skin. The resulting markings on the patient's skin can be subsequently used by a surgeon to identify the location of skin supplying blood vessels, such that he can avoid severing or otherwise damaging during a surgical procedure.

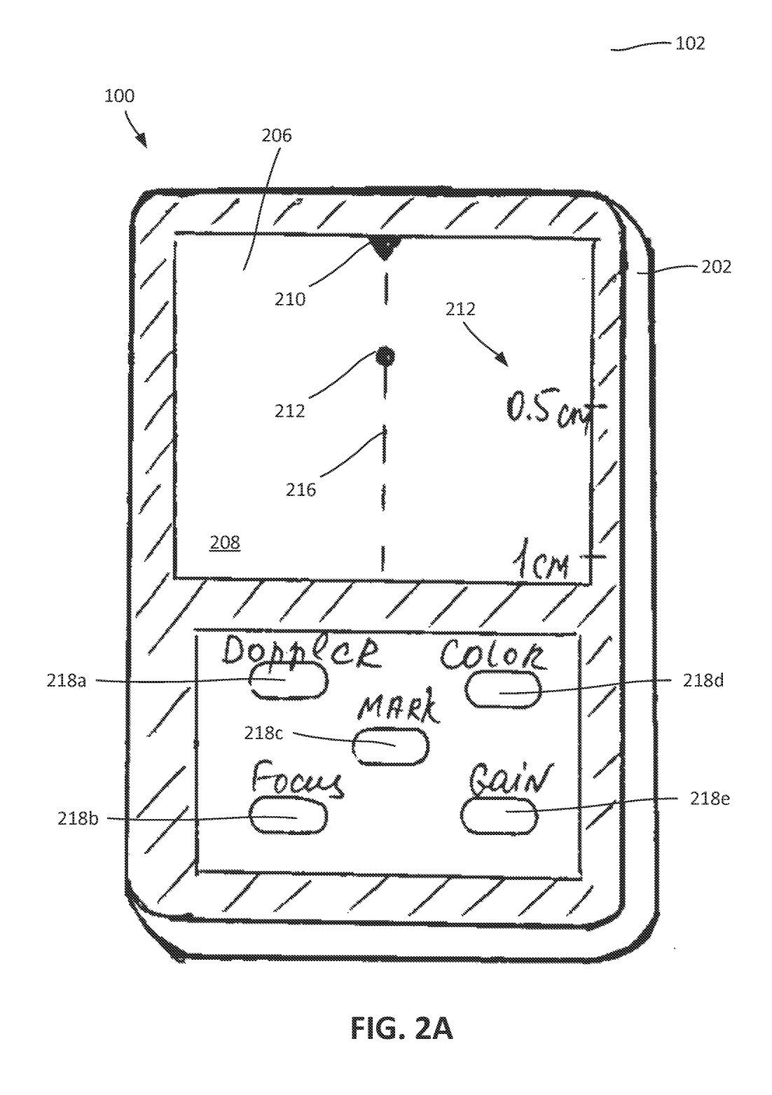

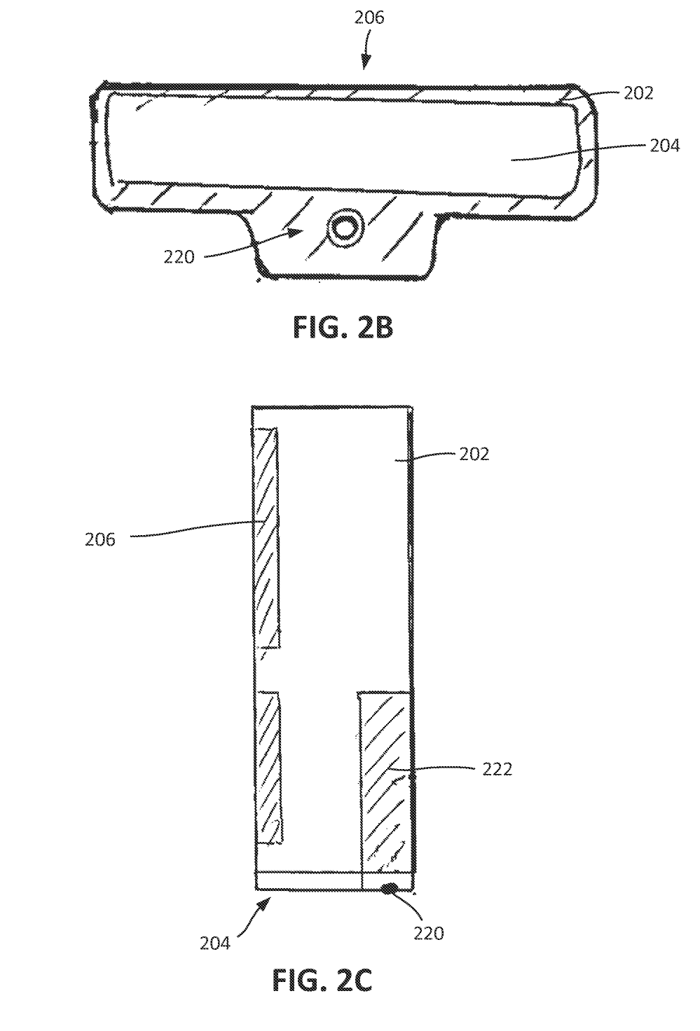

The system 100 depicted in FIG. 1 can be implemented in a variety of ways. For example, in some implementations, the system 100 can be implemented as a single hand-held device. FIGS. 2A-C shows a front view (FIG. 2A), bottom view (FIG. 2B), and side view (FIG. 2C) of an example hand-held implementation of system 100. Here, the system 100 includes a housing 202 that encloses each of the modules of system 100 (i.e., the imaging module 110, the display module 120, the marking module 130, and the processing module 140).

In this example, the system 100 includes a transducer 204 mounted to the bottom of the housing 202. The transducer 204 is a component of the imaging module 110, and sends pulses of ultrasonic energy into a region of the human body, and detects energy that returns from the region to the transducer 204. For example, the transducer 204 can be placed against the skin of a patient above a particular region of interest (e.g., against a particular portion of the patient's breast). Using the transducer 204, the imaging module 110 can transmit energy into that portion of the patient's skin and breast and generate images that depict that region.

In this example, the system 100 includes a display screen 206 mounted to the front of the housing 202. The display screen 206 is a component of the display module 120, and displays information to the user. For example, as shown in FIG. 2A, the display screen 206 displays an ultrasonic image 208, an alignment cursor 210, and scaling information 212. In this example, the ultrasonic image 208 depicts a blood vessel 214 positioned at a particular depth (as indicated by the scaling information 212), and is aligned with the cursor 210 (e.g., as indicated by the dotted line 216 that extends from the cursor 210). In some implementations, the display screen 206 can be an LCD screen, LED screen, OLED screen, or any other component that can be used to electronically display visual information.

The system 100 also includes several buttons 218a-e positioned on the front of the housing 202. The buttons 218a-e are operatively coupled to processing module 140, and allow the users to issue commands to the system 100. For example, the user can press the button 218a to specify one or more Doppler imaging modes (e.g., to obtain images that depict the blood flow). As another example, the user can press the button 218b to specify imaging focus-related settings. As another example the user can press the button 218c to indicate that the system 100 is aligned with a blood vessel and indicate that the marking module 130 can mark the skin. As another example, the user can press the button 218d to specify a particular color modes (e.g., to display the image 208 according to different color schemes). As another example, the user can pressed the button 218e to specify gain-related settings. Although example buttons 218a-e are described above, these are merely examples. Other buttons 218 can be used either in addition to or instead of the buttons 218a-e shown here. In some implementations, the screen 206 can detect physical inputs from a user and can be used instead of or in addition to the buttons 218a-e. For example, in some implementations, the screen 206 is a touch screen (e.g., a resistive or capacitive touch screen) that can detect when and where a user touches the screen 206. In these implementations, the screen 206 can display graphical elements that allow the user to view and select options as desired.

In this example, the system 100 also includes an ink applicator 220 mounted to the bottom of the housing 202. The ink applicator 220 is a component of the marking module 130, and applies ink to the surface of the patient's skin. In some implementations, the ink applicator 220 can be a retractable ink applicator that extends from the housing 202 to mark the skin, and retracts after marking is complete. In another example, the ink applicator 220 can be an applicator that releases ink selectively to mark the skin (e.g., using a pump), and stops releases ink after marking is complete. In the example shown here, the ink applicator 220 is positioned at a single point along the housing 202, and can be used to mark a single point along the patient's skin. In some implementations, the ink applicator 220 can be a part of a cartridge assembly that can be attached and removed from the housing 202 (e.g., to replace the ink in the ink applicator 220). For example, as shown in FIG. 2C, the ink applicator 220 is a part of a cartridge assembly 222 that can be detachably removed from the rear of the housing 202.

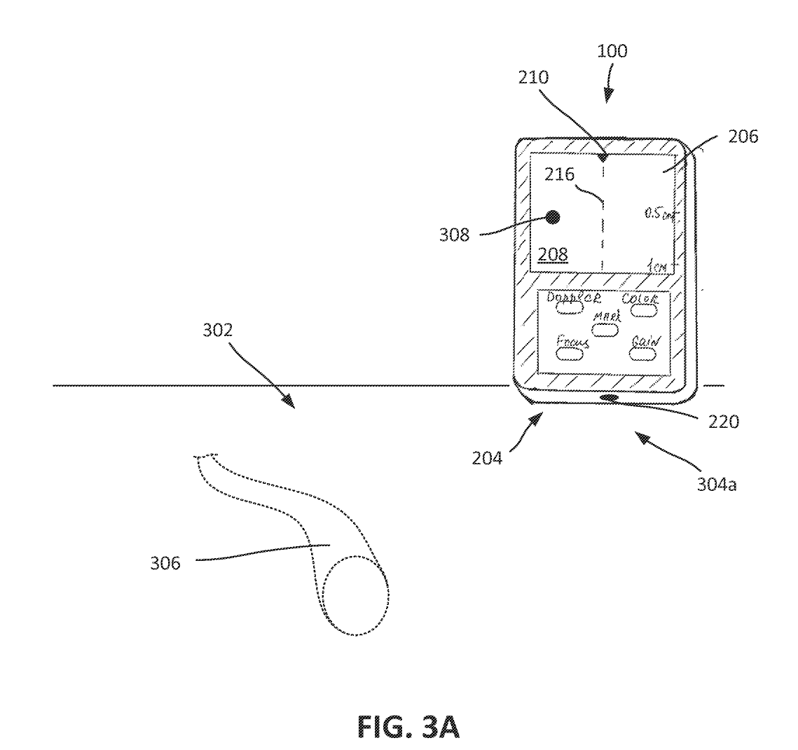

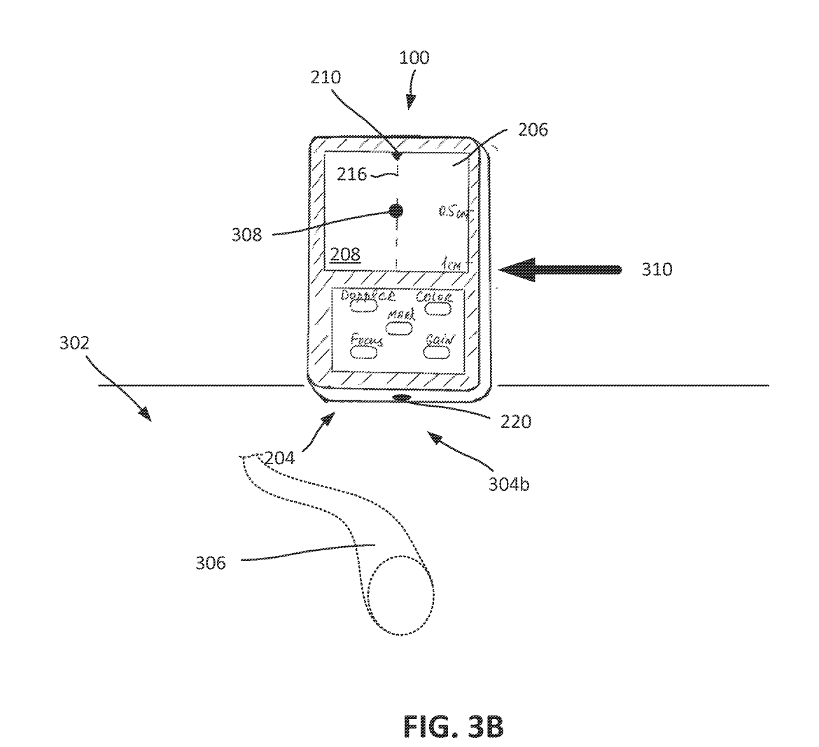

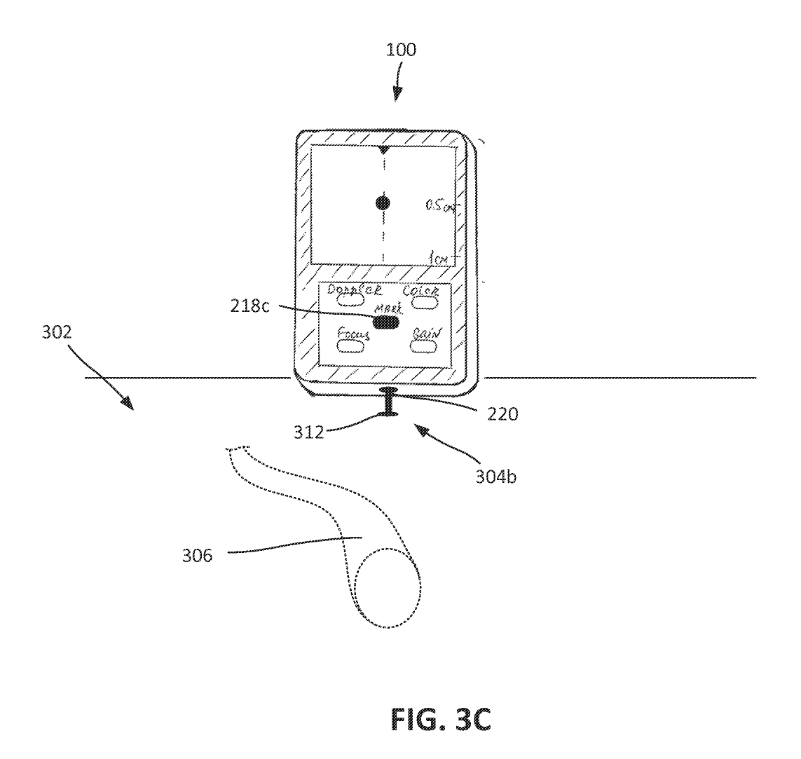

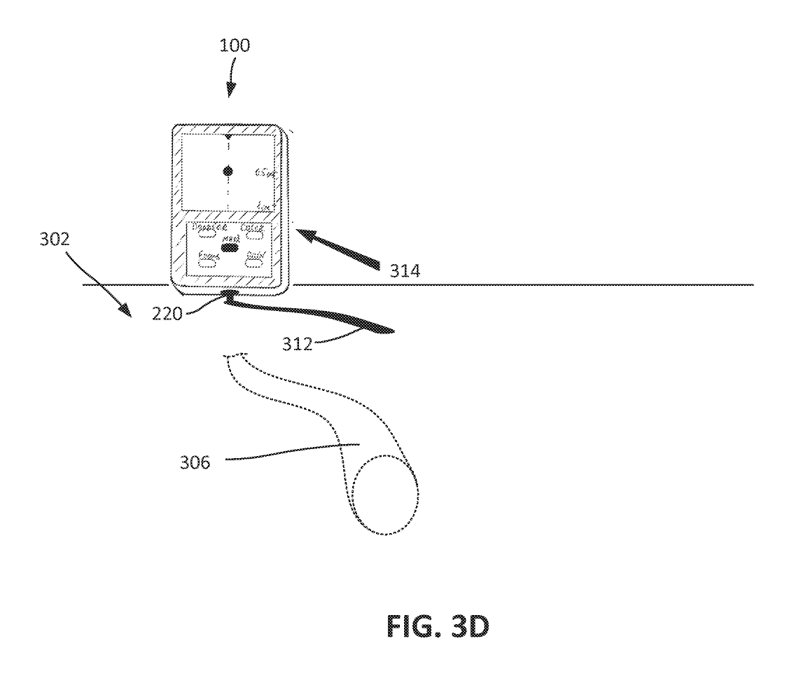

FIGS. 3A-D show an example usage of the example system 100 shown in FIGS. 2A-C. Referring to FIG. 3A, the user positions the system 100 against the surface 302 of a patient's skin at a point 304a, (e.g., such that the transducer 204 contacts the surface 302). The user then initiates imaging of the patient (e.g., by pressing an appropriate button 218 or selecting an appropriate command using the display screen 206 and the display module 120). The system 100 begins imaging the patient (e.g. using the transducer 204 and the other components of the imaging module 110), and generates ultrasonic images 208 of the portion of the patient surrounding the point 304a. The system 100 displays the images 208 to the user using the display module 120 (e.g., by presenting the images 208 on the display screen 206). In the example shown in FIG. 3A, the point 304a is close, but not directly atop a blood vessel 306 beneath the surface 302. Correspondingly, the image 208 includes a depiction 308 of the blood vessel 306 (e.g., a hypoechoic tubular structure) that is out of alignment with the cursor 210 and the dotted line 216. Thus, the ink applicator 220 of the marking module 130 is not positioned atop the blood vessel 306.

Using the display screen 206, the user observes that the depiction 308 of the blood vessel 306 is out of alignment with the cursor 210 and the dotted line 216. Referring to FIG. 3B, in response, the user moves the system 100 to along the surface 302 of the patient's skin to a point 304b (e.g., in the direction of arrow 310). During this time, the system 100 continues to generate images of the patient, and continuously updates the images 208 on the display screen 206. The images 208 can be updated in real-time or substantially real time. For example, in some cases, the between the transducer 204 transmitting ultrasonic energy and the corresponding image 208 being displayed on the display screen 206 is one second or less. In this manner, the user can use the display screen 206 to guide the movement of system 100. When the system 100 is positioned at point 304b, the depiction 308 of the blood vessel 306 (e.g., the hypoechoic tubular structure) is in alignment with the cursor 210 and the dotted line 216. Thus, the ink applicator 220 of the marking module 130 is positioned atop the blood vessel 306.

Referring to FIG. 3C, when the system 100 is aligned with the blood vessel 306, the user marks the point 304b using the marking module 130. In this example, the user presses the button 218c, which commands the marking module 130 to apply a mark 312 to the point 304b. As the point 304b is atop the blood vessel 306, the mark 312 indicates the presence of the blood vessel 306 below the surface.

As above, during this time, the system 100 continues to generate images of the patient, and continuously updates the images 208 on the display screen 206. Referring to FIG. 3D, using the display screen 206, the user moves the system 100 along the surface 302 while maintaining the alignment between the depiction 308 of the blood vessel 306, the cursor 210, and the dotted line 216 (e.g., in the direction of arrow 314). During this time, the marking module 130 continues applying ink to the surface 302. Thus, as the user continues to move the system 100 while maintaining the alignment between the depiction 308 of the blood vessel 306, the cursor 210, and the dotted line 216, the mark 312 is applied in a manner that traces the surface atop to the blood vessel 306.

Using the technique demonstrated in FIGS. 3A-D, the user can use the system 100 to identify a skin-supplying blood vessel near the surface of a user's skin, and mark that blood vessel for future reference. Further, the user can repeat this technique several times in order to mark multiple blood vessels. For example, after the user has marked a single blood vessel, the user can press the button 216c to discontinue marking, move the system 100 to a different location along the patient's body, and locate other blood vessels. When another blood vessel has been located, the user can align the system 100 with that blood vessel, and continue marking, as described above. In this manner, the user can identify and mark multiple vessels, as desired.

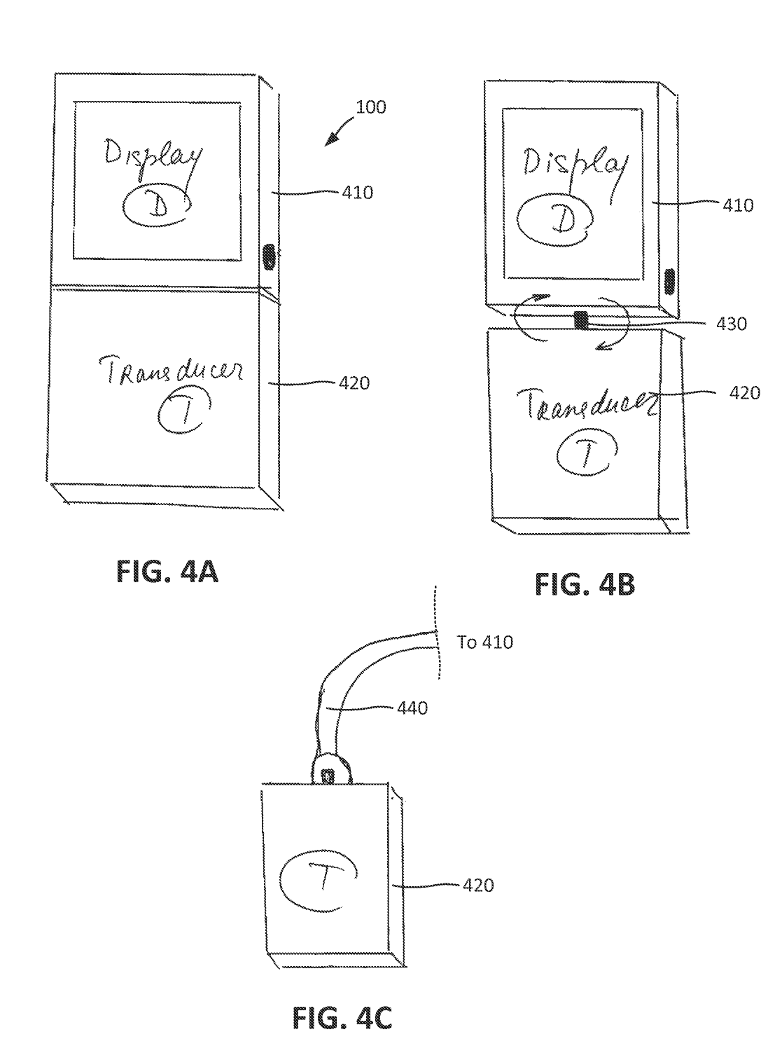

In the example implementation shown in FIGS. 2A-C, the system 100 includes a single housing 202, and each of the components of the system 100 (e.g., the imaging module 110, the display module 120, the marking module 130, and the processing module 140) are enclosed by the housing 202. However, this need not be the case. In some implementations, the system 100 can be implemented as a system of multiple interconnected devices. For example, referring in FIG. 4A, in some implementations, the 100 can include a first device 410 and a second device 420. The first device 410 includes a portion of the system 100 (e.g., the imaging module 110 and the marking module 130), while the second device 420 contains another portion of the system 100 (e.g., the display module 120 and the processing module 140). The devices 410 and 420 and operatively connected (e.g., through a wired or wireless data connection), such that the devices 410 and 420 can communicate. In some implementations, the devices 410 and 420 can be reversibly attached, such that a user can connect the devices 410 and 420 together as desired (e.g., prior to an imaging session), and disconnected the devices as desired (e.g., after an imaging session has been completed). In some implementations, the first device 410 can be a portable electronic device, such as a cellular phone, smartphone, or tablet computer. For example, a tablet computer can be used to display and process information, while a separate device 420 can be attached to the tablet computer (e.g., through a wired connector, a wireless connection, or a dock) to provide imaging and marking capabilities.

As another example, referring to FIG. 4B, in some implementations, the first device 410 can be connected to the second device 420 such that they can move relative to one another. For example, the devices 410 and 420 can be connected through a rotating joint 430 that allows one device to rotate relative to the other. In some circumstances, this can be beneficial, as it allows the imaging module 110 to be positioned relatively independently from the display module 120. Thus, in some cases, a user can position the imaging module 110 against the patient's skin, and rotate the display module 120 so that he can readily view the presented images. Although a rotating joint 430 is shown in the example above, this is merely an example. In some implementations, the system 100 can include a rotating joint, pivoting joint, a sliding joint, or combinations thereof.

In some implementations, the first device 410 and the second device 420 can be remote from each other (e.g., positioned several inches or feet inches from each other). In these implementations, the first device 410 and the second device 420 can be operatively connected through wired connector (e.g., a cable) or a wireless connection. For example, referring to FIG. 4C, the second device 420 can include a cable 440 that extends from the second device 420. This cable 440 can be connected and disconnected from the first device 410 as desired. For example, in some implementations, the first device 410 is a personal computer (e.g., a desktop computer or a laptop computer) that includes a display module 120 and a processing module 140, and the second device 420 includes an imaging module 110 and a marking module 130. In this example, the first device can be placed relatively distant from the second device 420, and can be connected through the cable 440. Thus, the components of system 100 need not all be in close proximity to the patients, and on or more of the components can be positioned relatively distant from the patient (e.g., on a counter, a desk, a table, a transport cart, or elsewhere).

In the examples above, each module of the system 100 is described as being distributed either in the first device 410 or the second device 420. However, in some implementations, a module can be implemented across both of the devices 410 and 420. For example, in some implementations, the first device 410 and the second device 420 can each include processing modules and collectively perform the operations described with respect to processing module 140. As another example, in some implementations, the first device 410 and the second device 420 can each include display devices, and can each perform the operations described with respect to display module 120.

In the example implementations devices above, the marking module 130 is configured to mark a single point along the patient's skin. Thus, if the user wishes to mark a blood vessel, he must manually align the system 100 such that it is aligned with the blood vessel (e.g., by maintaining the alignment between a cursor and the depiction of the blood vessel using the display module 120), then move the system 100 along the patient's skin such that this alignment is maintained. However, in some implementations, the system 100 can automatically (or semi-automatically) align itself with a blood vessel without requiring significant user input.

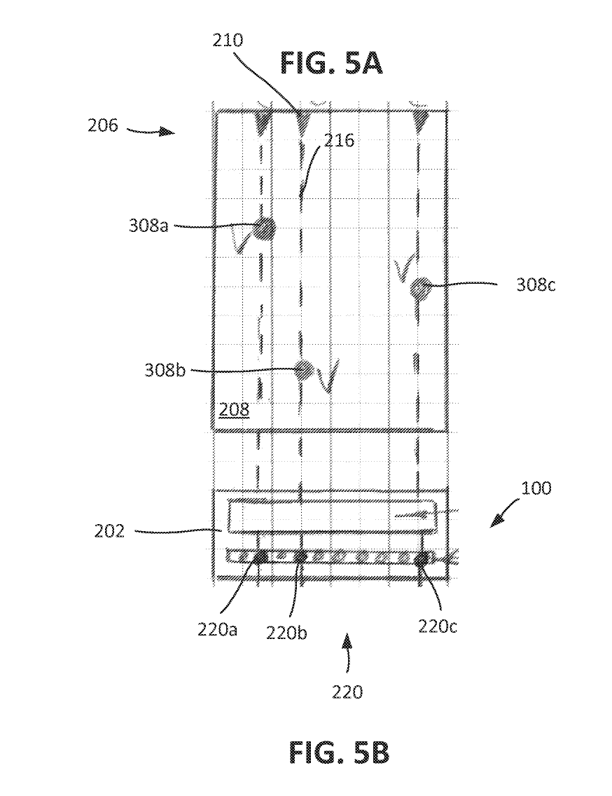

FIG. 5A shows an example implementation of a screen 206, and FIG. 5B shows the bottom of an example implementation of the system 100. In this example, the system 100 includes multiple ink applicators 220 positioned in a line across the bottom of the housing 202. During use, one or more of the ink applicators 220 can be used to apply ink to a patient's skin, depending on the images 208 displayed by the screen 206. For example, when the image 208 includes a depiction 308a of a blood vessel, the system 100 can activate a corresponding ink applicator 220a that aligns with the depiction 308a. As another example, when the image 208 includes a depiction 308b of a blood vessel, the system 100 can activate a corresponding ink applicator 220b that aligns with the depiction 308b. As another example, when the image 208 includes a depiction 308c of a blood vessel, the system 100 can activate a corresponding ink applicator 220c that aligns with the depiction 308c. In this manner, the system 100 need not be specifically aligned with respect to a particular blood vessel, and can instead be generally aligned with a particular blood vessel and activate an appropriate particular ink applicator.

In some implementations, the appropriate ink applicator can be selected manually. For example, in some cases, the user can move the cursor 210 and dotted line 216 (e.g., using a button or touch screen control) such that they align with a particular blood vessel. Correspondingly, a particular one of the ink applicators is selected and activated when appropriate. For example, as the user moves the system 100 along the surface of the patient's skin, the user can also move the cursor 210 and dotted line 216 to maintain alignment and apply markings in the correct locations.

In some implementations, the appropriate ink applicator can be selected automatically. For example, in some cases, the system 100 can analyze the images 208 in order to locate blood vessels. This can be performed in a variety of ways. As an example, the system 100 can obtain color Doppler images and detect regions of the images that correspond to flowing blood. These portions of the images can be segmented from the image 208, and identified as blood vessels. Based on this determination, the system 100 can selected a particular one of the ink applicator that aligns with the detected blood vessel, and activate that ink applicator when appropriate. For example, as the user moves the system 100 along the surface of the patient's skin, the system can automatically detect the location of the blood vessel, and activate the appropriate ink applicator and apply markings in the correct locations.

In some implementations, the system 100 can detect blood vessels based on particular criteria. For example, the system 100 can look for blood vessels at a particular depth below the surface. As another example, the system 100 can look for blood vessels having a particular diameter. These criteria can be user-defined and/or defined by the manufacturer, and can be adjusted in order to obtain the desired blood vessel detection behavior.

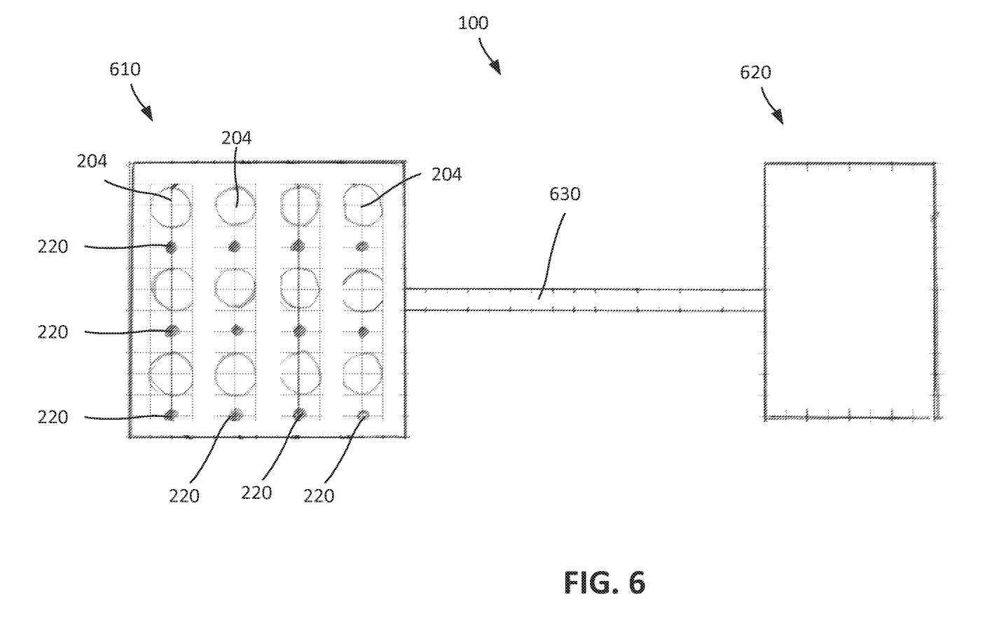

In some implementations, the ink applicators 220 can be positioned in a two dimension pattern, such that the markings can be applied without requiring that the system 100 be moved along the patient's skin. For example, as shown in FIG. 6, an implementation of the system 100 can include a first device 610 and a second device 620. The first device 610 is includes multiple ink applicators 220 and multiple transducers 204 positioned in a two dimensional pattern. The device 610 is flexible, such that the device 610 can conform to the patient's skin. For example the device 610 can include a flexible housing (e.g., a housing made of cloth, rubber, or other flexible material), with ink applicators 220 and transducers 204 mounted along the surface of the flexible housing. The device 610 is connected (e.g., through a cable 630) to the second device 620. The second device includes a display module 120 and a processing module 140. In a similar manner as described above, the system 100 can use an imaging module 110 (e.g., a transducer 204) to obtain images of a particular region of the patient's body, then mark the surface of the skin that is above a blood vessel. However, here, the system 100 includes multiple transducers 204 and ink applicators 220, and can thus can image and apply markings within a two-dimensional region without being moved. Thus, multiple portions of the patient can be image at once, and multiple points can be marked in accordance with the identified blood vessels. In an example usage, the first device 610 can be placed such that each of the ink applicators 220 and transducers 204 are flush against the patient's skin. The system 100 then images the patient, identifies blood vessels, and marks the location of the identified blood vessels on the patient's skin. Thus, the system 100 need not be moved in order to mark the blood vessels in a particular area of the patient's body. Although FIG. 6 shows an example implementation of the system 100, this is merely an example. In practice, the system 100 can include any number of ink applicators 220 and any number of transducer 204. Similarly, in practice, the arrangement of the ink applicators 220 and transducer 204 can also vary, depending on the application.

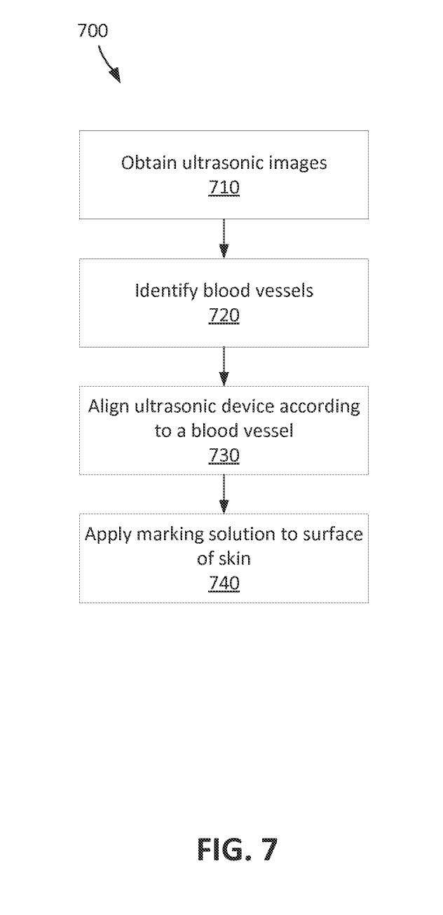

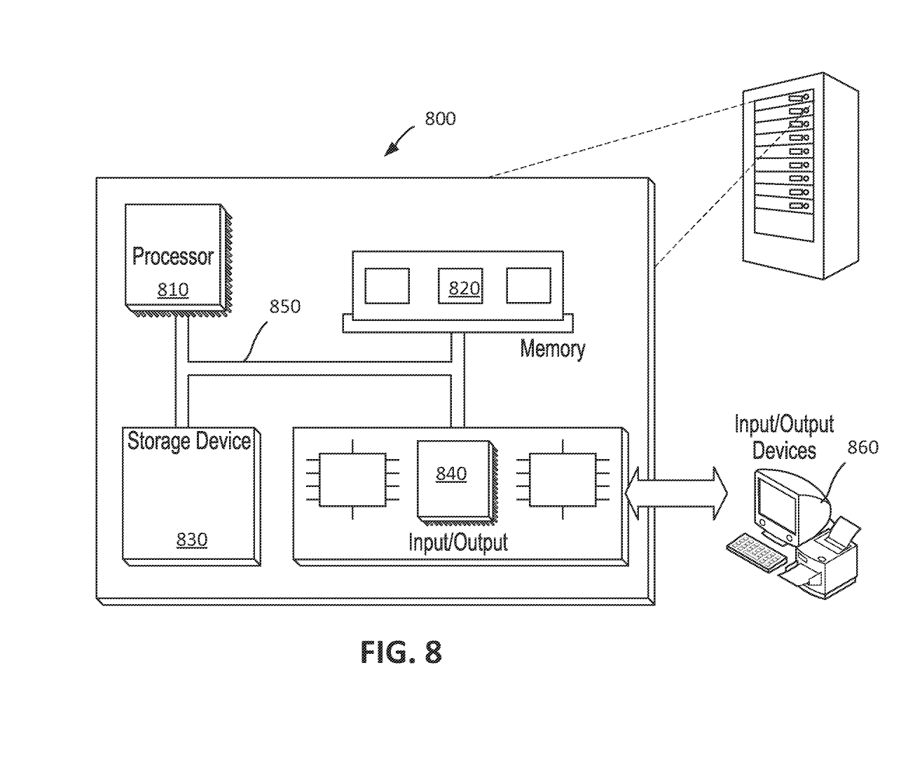

An example process 700 of detecting blood vessels is shown in FIG. 7. The process 700 begins by obtaining ultrasonic images corresponding to a part of the human body being examined (e.g., a portion of the human breast) (step 710). Ultrasonic images can be obtained using implementations of the system 100 described above. For example, a user can place a system 100 against a patient's skin, then initiate imaging of the patient using the system 100. As described above, the system 100 can generate ultrasonic energy, transmit this energy into a region of interest, and detect energy that returns to the imaging module 110. The system 100 can then determine ultrasonic images based on the detected energy. In some implementations, the system 100 can obtain images in real-time or substantially real time.

After obtaining ultrasonic images, the process 700 continues by identifying one or more blood vessels depicted in the ultrasonic images (step 720). As described above, this can be performed manually by a user, or semi-automatically or automatically by an ultrasonic device. For example, a system 100 can display the images using a display module 120, and the user can manually identify the location of blood vessels on the images. As another example, the system 100 can automatically detect the location of blood vessels in the images (e.g., by analyzing the images and automatically segmenting portions of the image corresponding to blood vessel-like structures). As another example, the system 100 can detected location of blood vessels based, at least in part, on criteria (e.g., user-defined criteria or manufacturer-defined criteria). In some implementations, the images are B-mode ultrasonic images, color Doppler images, or both. For example, the images can be B-mode color Doppler images that depict as blood vessels as colored objects (e.g., colored circles, ovals, ellipses, or other tubular structures), based on their flow pattern.

After identifying one or more blood vessels, the process 700 continues by aligning the ultrasonic imaging device according to a particular blood vessel identified in the ultrasonic images (step 730). As described above, this can be performed manually by the user, or semi-automatically or automatically by an ultrasonic device. For example, the user can determine the alignment of the ultrasonic imaging device with a particular blood vessel (e.g., by viewing the ultrasonic images on the display module 120 and comparing the location of the blood vessel against an alignment indicator, such a cursor). The user can then manually adjust the position of the system 100 relative to the patient until the system 100 is aligned with the blood vessel, or adjust the position of the cursor until it aligns with the blood vessel depicted in the images. As another example the system 100 can automatically determine the position of a blood vessel within an image, then determine if the system 100 (e.g., at least one of the ink applicators 220) is in alignment with the blood vessel. If not, the system 100 can prompt the user to move the system 100, such that it is aligned.

After aligning the ultrasonic imaging device according to a particular blood vessel, the process 700 continues by applying a marking substance (e.g., a marking solution such as ink, or a solid or semi-solid marking substance) to the surface of the patient's skin (step 740). As described above, this can be performed manually by the user, or semi-automatically or automatically by an ultrasonic device. For example, the user can determine that the system 100 is aligned with a blood vessel, then manually initiate marking using the marking module 130. As another example the system 100 can automatically determine that is in alignment with the blood vessel, and automatically initiate marking using the marking module 130.

In some implementations, the user can move the ultrasonic imaging device to a new location in order to obtain additional ultrasonic images corresponding to a different portion of the patient. In some cases, the user can move the ultrasonic imaging device such that the alignment between the ultrasonic imaging device and the identified blood vessel are maintained during movement. In this manner, the marking substance is applied to the patient along a path immediately above the identified blood vessel. In some implementations, the user need not manually maintain this alignment. For example, in some cases, the ultrasonic imaging device can determine the location of a blood vessel, then selectively activate one of several ink applicators according to the determined position.

In some implementations, some or all of the process 700 can be repeated in order to mark multiple blood vessels.

Implementations of process 700 can be performed as a part of a surgical procedure, as a mastectomy. For example, the process 700 can be performed on a patient's breast on around the nipple-areola complex in order to mark skin-supply blood vessels in the area (e.g., blood vessels that are near the surface of the skin, such as those approximately 1 cm or less from the surface of the skin). After the blood vessels have been marked, a user can conduct a surgical procedure in a manner that avoids the marked regions. For example, in some implementations, a surgeon can perform an incision on the skin of the patient (e.g., one or around the nipple-areola complex) such that the marked blood vessels are avoided. Thus, access beneath the skin is provided in a safe manner.

Some implementations of subject matter and operations described in this specification can be implemented in digital electronic circuitry, or in computer software, firmware, or hardware, including the structures disclosed in this specification and their structural equivalents, or in combinations of one or more of them. For example, in some implementations, imaging module 110, display module 120, marking module 130, and processing module 140 can be implemented using digital electronic circuitry, or in computer software, firmware, or hardware, or in combinations of one or more of them. In another example, processes 700 can be implemented, at least in part, using digital electronic circuitry, or in computer software, firmware, or hardware, or in combinations of one or more of them.

Some implementations described in this specification can be implemented as one or more groups or modules of digital electronic circuitry, computer software, firmware, or hardware, or in combinations of one or more of them. Although different modules can be used, each module need not be distinct, and multiple modules can be implemented on the same digital electronic circuitry, computer software, firmware, or hardware, or combination thereof.

Some implementations described in this specification can be implemented as one or more computer programs, i.e., one or more modules of computer program instructions, encoded on computer storage medium for execution by, or to control the operation of, data processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read only memory or a random access memory or both. A computer includes a processor for performing actions in accordance with instructions and one or more memory devices for storing instructions and data. A computer may also include, or be operatively coupled to receive data from or transfer data to, or both, one or more mass storage devices for storing data, e.g., magnetic, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks, and CD ROM and DVD-ROM disks. The processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

FIG. 9 shows an example computer system 800. The system 800 includes a processor 810, a memory 820, a storage device 830, and an input/output device 840. Each of the components 810, 820, 830, and 840 can be interconnected, for example, using a system bus 850. The processor 810 is capable of processing instructions for execution within the system 800. In some implementations, the processor 810 is a single-threaded processor, a multi-threaded processor, or another type of processor. The processor 810 is capable of processing instructions stored in the memory 820 or on the storage device 830. The memory 820 and the storage device 830 can store information within the system 800.

The input/output device 840 provides input/output operations for the system 800. In some implementations, the input/output device 840 can include one or more of a network interface devices, e.g., an Ethernet card, a serial communication device, e.g., an RS-232 port, and/or a wireless interface device, e.g., an 802.11 card, a 3G wireless modem, a 4G wireless modem, etc. In some implementations, the input/output device can include driver devices configured to receive input data and send output data to other input/output devices, e.g., keyboard, printer and display devices 860. In some implementations, mobile computing devices, mobile communication devices, and other devices can be used.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable subcombination.

A number of implementations have been described. Nevertheless, it will be understood that various modifications may be made without departing from the spirit and scope of the invention. Accordingly, other implementations are within the scope of the following claims.

What is claimed is:

1. A method for indicating blood vessels of a human body, the method comprising:
obtaining, using a handheld ultrasonic imaging device, ultrasonic images corresponding to a portion of the human body;
identifying, using a display of the ultrasonic imaging device, one or more blood vessels depicted in the ultrasonic images;
aligning the ultrasonic imaging device according to a particular blood vessel identified in the ultrasonic images; and
applying, using the ultrasonic imaging device, a marking substance to a surface of the human breast, wherein the marking substance is applied to a point immediately above the particular blood vessel.

2. The method of claim 1, wherein aligning the ultrasonic imaging device comprises moving the ultrasonic imaging device with respect to the human body until an indicator of the display aligns with the particular vessel identified in the ultrasonic images.

3. The method of claim 1, wherein aligning the ultrasonic imaging device comprises moving an indicator of the display with respect to the ultrasonic images until the indicator aligns with the particular vessel identified in the ultrasonic images.

4. The method of claim 1, further comprising:
moving the ultrasonic imaging device along the human body in order to obtain additional ultrasonic images corresponding to different portions of the human body;
maintaining the alignment between the ultrasonic imaging device and the particular blood vessel identified in the additional ultrasonic images during movement of the ultrasonic imaging device; and
applying, using the ultrasonic imaging device, the marking substance onto the surface of the human body, wherein the marking substance is applied along a path immediately above the particular blood vessel.

5. The method of claim 1, wherein the ultrasonic images are displayed in real-time on the display of the ultrasonic imaging device.

6. The method of claim 1, further comprising:
repeating the steps of obtaining, identifying, aligning, and applying until a plurality of blood vessels of the human body are marked.

7. The method of claim 6, further comprising incising the human body in a manner that avoids the applied marking substance.

8. The method of claim 7, wherein incising the human breast is performed as a part of a mastectomy procedure.

9. The method of claim 1, wherein the particular blood vessel is a skin-supplying blood vessel.

10. The method of claim 1, wherein the particular blood vessel is within approximately 1 cm of a surface of the human body.

11. The method of claim 1, wherein the ultrasonic images are B-mode, color Doppler ultrasonic images.

12. A ultrasonic imaging system for indicating blood vessels of a human body, the system comprising:
an ultrasonic imaging module;
a display module;
a marking module; and
processing module operatively coupled to the ultrasonic imaging module, the display module, and the marking module;
wherein the processing module is configured to:
obtain, using the ultrasonic imaging module, ultrasonic images corresponding to a portion of the human body; and
present, using the display module, the ultrasonic images, wherein the ultrasonic images depict one or more blood vessels of the human body;
determine that the ultrasonic imaging system is aligned according to a particular blood vessel depicted in the ultrasonic images; and
upon determining that the ultrasonic imaging system is aligned according to the particular blood vessel depicted in the ultrasonic images, apply, using the marking module, a marking substance to a surface of the human body,
wherein the marking substance is applied to a point immediately above the particular blood vessel.

13. The system of claim 12, wherein determining that the ultrasonic imaging system is aligned according to the particular blood vessel depicted in the ultrasonic images comprises:
receiving user input indicating that the particular blood vessel is aligned with an alignment indicator of the display module.

14. The system of claim 13, wherein the alignment indicator comprises a cursor.

15. The system of claim 13, wherein the cursor is configured to move with respect to the ultrasonic images.

16. The system of claim 12, wherein determining that the ultrasonic imaging system is aligned according to the particular blood vessel depicted in the ultrasonic images comprises:
identifying, using the processing module, one or more blood vessels depicted in the ultrasonic images;
determining that the ultrasonic imaging system is aligned according to the particular blood vessel based on the identification.

17. The system of claim 16, wherein identifying one or more blood vessels depicted in the ultrasonic images comprises identifying regions of the ultrasonic images indicative of flowing blood.

18. The system of claim 16, wherein identifying one or more blood vessels depicted in the ultrasonic images comprises receiving user input indicating a location of a blood vessel.

19. The system of claim 12, wherein the marking module comprises an applicator configured to apply the marking substance at a pre-defined point relative to the ultrasonic imaging system.

20. The system of claim 12, wherein the marking module comprises a plurality of applicators each configured to apply the marking substance at a respective pre-defined point relative to the ultrasonic imaging system.

21. The system of claim 21, wherein the pre-defined points is arranged as a line.

22. The system of claim 22, wherein the plurality of pre-defined points is arranged as a grid.

23. The system of claim 20, wherein applying a marking substance to a surface of the human body comprises selecting a particular applicator to apply the marking substance based on an alignment between the particular applicator and the particular blood vessel.

24. The system of claim 12, wherein the processor is configured to present the ultrasonic images in substantially real-time.
